(19) United States
(12) Patent Application Publication
(10) Pub. No.: US 2026/0166009 A1
(43) Pub. Date: Jun. 18, 2026

(54) METHOD OF PREVENTING AND/OR TREATING TISSUE DAMAGE OR INJURY

(71) Applicant: MOREHOUSE SCHOOL OF MEDICINE, Atlanta, GA (US)

(72) Inventors: Julia LIU, Decatur, GA (US); Sudip BANERJEE, Atlanta, GA (US)

(21) Appl. No.: 19/419,490

(22) Filed: Dec. 15, 2025

Related U.S. Application Data
(60) Provisional application No. 63/734,376, filed on Dec. 16, 2024.

(51) Int. Cl. A61K 31/405; A61K 38/49; A61K 45/06; A61P 1/00

(52) U.S. Cl. CPC A61K 31/405; A61K 38/49; A61K 45/06; A61P 1/00; C12Y 304/21068

(57) ABSTRACT

A method of preventing and/or treating gastrointestinal damage in a subject comprising: administering in a therapeutically effective amount a pharmaceutical composition comprising a PAI-1 inhibitor to a subject having gastrointestinal damage. A method of preventing and/or treating caustic esophageal strictures in a subject comprising: administering in a therapeutically effective amount a pharmaceutical composition comprising a PAI-1 inhibitor to a subject having caustic gastrointestinal damage.

This application claims the priority of U.S. Provisional Application No. 63/734,376, filed on Dec. 16, 2024, which is incorporated herein by reference in its entirety.

### FIELD

The present application generally relates to medical treatment and in particular, a preventative treatment of injury in the gastrointestinal tract.

### BACKGROUND

Caustic substances are found in many common household products, e.g., oven cleaners that contain sodium hydroxide (lye) or potassium hydroxide, drain cleaners that contain sulfuric acid, hydrochloric acid, or lye, toilet bowl cleaners that contain hydrochloric acid or bleach (sodium hypochlorite), and rust removers that contain oxalic acid or phosphoric acid. Moderately caustic items can include: ammonia-based cleaners, bleach, detergents, hair relaxers, or button batteries. When swallowed, caustic substances can also burn the tongue, mouth, esophagus, and stomach. These burns may cause perforations (holes) of the esophagus or stomach. Food and saliva leaking from a perforation cause severe, sometimes deadly infection within the chest (mediastinitis or empyema) or abdomen (peritonitis). Accidental or intentional ingestion of caustic materials can result in devastating esophageal injuries including caustic esophageal strictures (CES), a debilitating condition without effective treatment. A single exposure to caustic agents such as lye or bleach (common house cleaning products) is an immediate medical emergency; ˜10% of these exposures lead to formation of caustic esophageal strictures (CES) in adults. CES is a lifelong clinical condition that is difficult to manage, and complicated by the development of squamous esophageal cancer, in up to 30% of CES patients. CES afflicts around 500 adults annually in the United States fitting the FDA criteria for a rare disease. The most recent estimate for the prevalence of adults with CES in the United States is based on a retrospective analysis of 40,844 Emergency Department (ED) visits over a 5-year span (2010-2014) for caustic ingestions, i.e. approximately 8,000 per year. Of these ED visits, the 1,500 patients were hospitalized, with up to a third of hospitalized patients developing CES. The number of pediatric CES cases per year in the US has not been evaluated in a similar manner. Ingestions of household cleaning products is the most common toxic exposure in young children, accounting for approximately 11% of the 230,000 reported toxic exposures per year. Most of ingestions in young children are accidental, while ingestions in adolescents and adults tend to be intentional and represent suicide attempts. Based on these data, the estimate for the yearly prevalence of CES resulting from toxic exposures in children five years or younger is comparable magnitude to adults. Thus, CES affects around 1,000 adults and children annually.

Caustic esophageal stricture (CES) is a rare disease affecting approximately 1,000 adults and children in the United States annually. Despite intense research into the pathogenesis of CES for over a century, no effective treatments have been found. Extensive investigation into the pathogenesis of CES in rabbit and rat models failed to yield any meaningful new therapies or therapeutic targets. The lack of effective medical therapy makes the management of caustic esophageal injuries and CES challenging in both adults and children. Numerous empiric therapies such as corticosteroids, broad-spectrum antibiotics, acid suppression therapies have not been beneficial in patients with caustic ingestions. Thus, there is currently no FDA approved medical treatment for caustic ingestion induced esophageal injuries and CES. For patients with CES, the only definitive symptomatic treatment is esophagectomy with colonic interposition, which does eliminate the risk of caustic ingestion related esophageal cancer.

While esophageal strictures are more frequent, stomach strictures are still a significant concern. Some studies indicate that stomach strictures occur in about 10-20% of patients with severe caustic injuries. Alkaline substances (like lye) are more likely to cause stomach strictures than acidic substances. Prompt medical care can reduce the severity of damage and the likelihood of strictures. Caustic tongue burns arise from contact with corrosive substances, leading to chemical injury characterized by pain, erythema, edema, and potentially blistering. The severity correlates with the substance's concentration, contact duration, and quantity. While less frequent than esophageal or gastric strictures due to reflexive spitting and salivary dilution, tongue burns can heal with fibrotic scarring, causing strictures that restrict mobility and impair speech, swallowing, and even respiration. These strictures, though less prevalent than those in the digestive tract, necessitate prompt medical intervention to mitigate long-term functional deficits and address potential airway compromise.

### SUMMARY

An aspect of the present application is related to a method of preventing and/or treating gastrointestinal damage in a subject, comprising the step of administering to the subject an effective amount of a pharmaceutical composition comprising a plasminogen activator inhibitor-1 (PAI-1) activity modifier.

An aspect of the present application relates to a method for reducing gastrointestinal inflammation in a subject, comprising the step of administering to the subject an effective amount of a pharmaceutical composition comprising a PAI-1 activity modifier.

An aspect of the present application relates to a method for reducing gastrointestinal scar formation and loss of function in a subject, comprising the step of administering to the subject an effective amount of a pharmaceutical composition comprising a PAI-1 activity modifier.

An aspect of the present application relates to a method for rejuvenating or reconstituting damaged tissue in a subject suffering from caustic injury, comprising the step of administering to the subject an effective amount of a pharmaceutical composition comprising a PAI-1 activity modifier.

An aspect of the present application relates to a method for preventing or treating a stricture in a subject, comprising the step of administering to the subject an effective amount of a pharmaceutical composition comprising a PAI-1 activity modifier.

These and other aspects and embodiments of the present application will become better understood with reference to the following detailed description when considered in association with the accompanying drawings and claims.

### BRIEF DESCRIPTION OF DRAWINGS

An understanding of the features and advantages of the present application will be obtained by reference to the following detailed description that sets forth illustrative embodiments, in which the principles of the application may be utilized, and the accompanying drawings. The figures herein are for illustrative purposes only and are not necessarily drawn to scale.

FIG. 1A shows experimental paradigm of oral alkali exposure and therapeutic application of small molecule PAI-1 inhibitor.

FIG. 1B shows change in relative mouse weight during the experiment (n=12 Control, 6 of Control+PAI-1 inhibitor, 17 of Alkali only, 5 of Alkali+tiplaxtinin, 12 Serpine1+/−, 8 Serpine1+/−).

FIG. 1C shows radiographs of the thoracic cavity of mice at 7-days following alkali exposure, with oral Barium contrast administration. Red arrows denote areas of stricture in the esophagus, i.e. CES, present in 16/25 (64%) of Alkali only mice, 0/5 Alkali+tiplaxtinin treated mice, 2/12 (17%) Serpine1+/−+alkali and 0/8 Serpine1+/−+Alkali.

FIG. 2A shows representative inverted grayscale photomicrographs of anti-PAI-1 immunofluorescence staining of esophageal sections from mice treated as indicated (2 to 4 mice per group×2 to 4). Note stronger signal in both smooth muscle (orange arrow) and epithelial layer (blue arrowhead) 7 days following alkali instillation. Antibody specificity was confirmed by lack of signal in Serpine1−/− esophageal tissue.

FIG. 2B shows representative fluorescence micrographs of anti-Desmin stained esophageal sections from WT and Serpine1−/− mice treated as indicated (2 to 4 mice per group×2 to 4). Images are shown with range indicator lookup table to demonstrate variation and patterns of signal intensity.

FIG. 3 shows principal component analysis (PCA) of the control and lye-treated groups.

FIG. 4 shows a volcano plot of genes that are significantly altered between the control and lye-treated groups.

FIG. 5 shows the fold-change in PAI-1 gene expression between the control and lye-treated groups.

FIG. 6 shows enrichment of biological pathways in control and lye-treated mice in response to PAI-1 expression.

### DETAILED DESCRIPTION

The aspects of the application are described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

As used in this specification and the appended claims, the singular forms “a,” “an” and “the” include plural referents unless the content clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

The recitation of numerical ranges by endpoints includes all numbers and fractions subsumed within the respective ranges, as well as the recited endpoints.

As used herein “downregulated,” “reduced expression,” “underexpressed,” and variations thereof refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a breast tumor and comprises breast tissue, said control is breast tissue of a healthy individual. The term “reduced expression” preferably refers to at least a 10% reduction, e.g., at least a 15%, 20%, 25%, 30%, 40%, 50%, 60%, 70%, 75%, 80%, 85%, 90%, 95%, 98% or 99% reduction, relative to such control.

As used herein “upregulated,” “increased expression,” “overexpressed,” and variations thereof are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein, the specific mention of a particular gene or gene product includes species homologs of that gene or gene product.

PAI-1 (also known as serpine1 and encoded by the SERPINE1 gene in humans) acts as a central node in the global transcriptomic responses of epithelial and myeloid inflammatory cells following acute intestinal injury. PAI-1 is a target that was identified as an important link between the epithelial and myeloid cells in the context of inflammatory bowel disease (IBD), and its expression is elevated in response to injury throughout the gastrointestinal tract. PAI-1 is a member of serine protease inhibitor (SERPIN) family that acts as the primary inhibitor of two main mammalian plasminogen activators, urokinase-type (uPA; also known as urokinase or urinary-type) and tissue-type (tPA). As the main negative regulator of plasminogen activation, PAI-1 is an essential factor in regulating the physiological balance between thrombosis and fibrinolysis. PAI-1 is a labile molecule that exists in four different forms: active, latent, cleaved and target bound form. High PAI-1 levels inhibit fibrinolysis (clot breakdown) and are associated with increased risk of cardiovascular conditions, such as thrombophilia, heart disease, stroke, diabetes, obesity, severe COVID-19, coronary artery disease, deep vein thrombosis, and pulmonary embolism. PAI-1 also plays important role in cell migration, adhesion, senescence, cancer invasion and tissue remodeling. Moreover, the PAI-1 level was extensively validated as the biological prognostic factor in breast cancer and as a marker of a poor prognosis in other cancers. PAI-1 is also one of the plasma biomarkers associated with nonalcoholic fatty liver disease.

The term “PAI-1 activity modifier,” as used hereinafter, refers to an agent that is capable of binding to a PAI-1 and reducing the activity of PAI (such as a PAI inhibitor) or that is capable of binding to a PAI-1 and reducing the amount of unbound PAI-1 (such as tPA, recombinant tPA (rtPA), uPA and recombinant uPA (ruPA)).

The term “treatment,” as used herein, refers to any type of therapy, which aims at terminating, preventing, ameliorating or reducing the susceptibility to a clinical condition as described herein. In a preferred embodiment, the term treatment relates to prophylactic treatment (i.e. a therapy to reduce the susceptibility to a clinical condition), of a disorder or a condition as defined herein. Thus, “treatment,” “treating,” and their equivalent terms refer to obtaining a desired pharmacologic or physiologic effect, covering any treatment of a pathological condition or disorder in a mammal, including a human. The effect may be prophylactic in terms of completely or partially preventing a disorder or symptom thereof and/or may be therapeutic in terms of a partial or complete cure for a disorder and/or adverse effect attributable to the disorder. That is, “treatment” includes (1) preventing the disorder from occurring or recurring in a subject, (2) inhibiting the disorder, such as arresting its development, (3) stopping or terminating the disorder or at least symptoms associated therewith, so that the host no longer suffers from the disorder or its symptoms, such as causing regression of the disorder or its symptoms, for example, by restoring or repairing a lost, missing or defective function, or stimulating an inefficient process, or (4) relieving, alleviating, or ameliorating the disorder, or symptoms associated therewith, where ameliorating is used in a broad sense to refer to at least a reduction in the magnitude of a parameter, such as inflammation, pain, or immune deficiency.

The terms “prevent,” “preventing,” or “prevention,” as used herein, refer to a method of barring a subject from acquiring a disorder and/or its attendant symptoms. In some embodiments, the terms “prevent,” “preventing,” or “prevention” refer to a method of reducing the risk of acquiring a disorder and/or its attendant symptoms.

“Patient” or “subject” as used herein means a mammalian animal, including a human, a veterinary or farm animal, a domestic animal or pet, and animals normally used for clinical research. In one embodiment, the subject of these methods and compositions is a human. In another embodiment, the subject is a female.

The term “inhibits” is a relative term, an agent inhibits a response or condition if the response or condition is quantitatively diminished following administration of the agent, or if it is diminished following administration of the agent, as compared to a reference agent. Similarly, the term “prevents” does not necessarily mean that an agent completely eliminates the response or condition, so long as at least one characteristic of the response or condition is eliminated. Thus, a composition that reduces or prevents an infection or a response, such as a pathological response, can, but does not necessarily completely eliminate such an infection or response, so long as the infection or response is measurably diminished, for example, by at least about 50%, such as by at least about 70%, or about 80%, or even by about 90% of (that is to 10% or less than) the infection or response in the absence of the agent, or in comparison to a reference agent.

The term “increased level” refers to a level that is higher than a normal or control level customarily defined or used in the relevant art. For example, an increased level of immunostaining in a tissue is a level of immunostaining that would be considered higher than the level of immunostaining in a control tissue by a person of ordinary skill in the art.

### Methods

One aspect of the present application relates to a method of preventing and/or treating tissue damage/tissue injury in a subject. The method comprises the step of administering to the subject an effective amount of a pharmaceutical composition comprising a PAI-1 activity modifier.

Another aspect of the present application relates to a method for ameliorating tissue damage/tissue injury and/or alleviating symptoms of tissue damage/tissue injury in a subject. The method comprises the step of administering to the subject an effective amount of a pharmaceutical composition comprising a PAI-1 activity modifier.

Another aspect of the present application relates to a method for improving tissue rejuvenation/reconstitution in a subject suffering from damage/tissue injury. The method comprises the step of administering to the subject an effective amount of a pharmaceutical composition comprising a PAI-1 activity modifier.

Another aspect of the present application relates to a method for reducing inflammation and/or scar formation in a subject suffering from tissue damage/tissue injury. The method comprises the step of administering to the subject an effective amount of a pharmaceutical composition comprising a PAI-1 activity modifier.

Another aspect of the present application relates to a method of preventing and/or treating caustic esophageal strictures in a subject. The method comprises the step of administering in an effective amount a pharmaceutical composition comprising a PAI-1 activity modifier.

Another aspect of the present application relates to a method of preventing and/or treating caustic tongue strictures in a subject, comprising the step of administering to the subject an effective amount of a pharmaceutical composition comprising a PAI-1 activity modifier.

Another aspect of the present application relates to a method of preventing and/or treating caustic stomach strictures in a subject, comprising the step of administering to the subject an effective amount of a pharmaceutical composition comprising a PAI-1 activity modifier.

### Tissue Damage/Tissue Injury

The tissue damage or tissue injury can be damage/injury of any tissue. In some embodiments, the tissue damage/tissue injury is gastrointestinal damage or gastrointestinal injury. In some embodiments, tissue damage/tissue injury is caustic gastrointestinal damage/injury. In some embodiments, the tissue damage/tissue injury is caustic gastrointestinal stricture. In some embodiments, the tissue damage or tissue injury is caustic tongue strictures. In some embodiments, the tissue damage or tissue injury is caustic esophageal strictures. In some embodiments, the tissue damage or tissue injury is caustic stomach strictures.

In some embodiments, the tissue damage or tissue injury is caustic intestinal strictures.

Caustic substances are found in many common household products, e.g., oven cleaners that contain sodium hydroxide (lye) or potassium hydroxide, drain cleaners that contain sulfuric acid, hydrochloric acid, or lye, toilet bowl cleaners that contain hydrochloric acid or bleach (sodium hypochlorite), and rust removers that contain oxalic acid or phosphoric acid. Moderately caustic items can include: ammonia-based cleaners, bleach, detergents, hair relaxers, or button batteries. When swallowed, caustic substances can also burn the tongue, mouth, esophagus, and stomach. These burns may cause perforations (holes) of the esophagus or stomach. Food and saliva leaking from a perforation cause severe, sometimes deadly infection within the chest (mediastinitis or empyema) or abdomen (peritonitis), which can sometimes lead to sepsis.

While esophageal strictures are more frequent, stomach strictures are also a significant concern. Some studies indicate that stomach strictures occur in about 10-20% of patients with severe caustic injuries. Alkaline substances (like lye) are more likely to cause stomach strictures than acidic substances. Prompt medical care can reduce the severity of damage and the likelihood of strictures. Caustic tongue burns arise from contact with corrosive substances, leading to chemical injury characterized by pain, erythema, edema, and potentially blistering. The severity correlates with the substance's concentration, contact duration, and quantity. While less frequent than esophageal or gastric strictures due to reflexive spitting and salivary dilution, tongue burns can heal with fibrotic scarring, causing strictures that restrict mobility and impair speech, swallowing, and even respiration. These strictures, though less prevalent than those in the digestive tract, necessitate prompt medical intervention to mitigate long-term functional deficits and address potential airway compromise.

Injuries of the gastrointestinal tract, such as strictures, can occur due to a variety of causes. In addition to caustic injury, injury of the esophagus, mouth, tongue, throat, stomach, and intestinal tract can be caused by thermal injury, such as esophageal thermal injury (ETI), due to extremely hot food or drink. Additionally, gastrointestinal tract injury can be the result of scarring from chronic acid reflux (GERD), radiation therapy, certain medications (such as NSAIDs, some antibiotics), inflammatory conditions like eosinophilic esophagitis, tumors, and congenital issues. Damage and inflammation lead to scar tissue, which contracts and tightens the esophagus.

### PAI-1 Activity Modifier

The PAI-1 activity modifier can be any agent that is capable of binding to PAI-1 and reducing the activity of PAI-1, such as a PAI-1 inhibitor, or any agent that is capable of binding to a PAI-1 and reducing the amount of unbound PAI-1, such as tPA, rtPA, uPA and ruPA, which may be considered as competitive PAI-1 inhibitors in a sense that they interact with PAI-1 and prevent the bound PAI-1 from inhibiting other plasminogen activators (PAS).

Plasminogen activators are serine proteases that trigger the conversion of plasminogen to plasmin, the central enzyme in fibrinolysis responsible for dissolving fibrin clots. This intricate process is vital for maintaining vascular patency, wound healing, and tissue remodeling. Key plasminogen activators include tPA and uPA, both existing in endogenous and recombinant forms (rtPA and ruPA, respectively). rtPA, notably alteplase, has revolutionized thrombolytic therapy for acute ischemic stroke, myocardial infarction, and pulmonary embolism by rapidly dissolving clots and restoring blood flow. While uPA finds clinical use in urinary tract thrombolysis and wound healing, other fibrinolytic agents contribute to this complex system. Streptokinase and staphylokinase, derived from bacteria, indirectly activate plasminogen, offering alternative thrombolytic strategies. Furthermore, kallikreins and Factor XIIa, components of the coagulation cascade, also possess plasminogen-activating capabilities. Intriguingly, vampire bat salivary plasminogen activator (Bat-PA) exhibits potent and prolonged fibrinolytic activity, holding promise for future therapeutic development. This diverse array of plasminogen activators highlights the intricate regulation of fibrinolysis and offers a range of therapeutic options for managing thrombotic conditions and promoting tissue repair. PAI-1 is the principal inhibitor of both t-PA and u-PA.

### PAI-1 Inhibitors

PAI-1 inhibitors block the action of PAI-1. Example of PAI-1 inhibitors include, but are not limited to, the following compounds and pharmaceutically acceptable salts thereof.

Tiplaxtinin (also known as tiplasinin or PAI-039) is a selective and orally efficacious inhibitor of PAI-1 with IC50 of 2.7 μM.

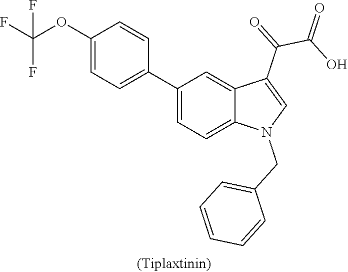 [SMILES: O=C(O)C(=O)c1cn(Cc2ccccc2)c2ccc(-c3ccc(OC(F)(F)F)cc3)cc12]

TM5441 is an orally bioavailable inhibitor of plasminogen activator inhibitor-1 (PAI-1), has IC50 values between 13.9 and 51.1 μM and induces intrinsic apoptosis in several human cancer cell lines. TM5441 attenuates Nω-nitro-1-arginine methyl ester-induced cardiac hypertension and vascular senescence.

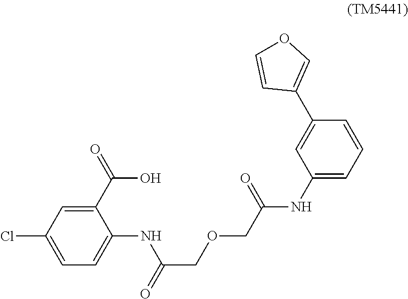 [SMILES: O=C(COCC(=O)Nc1ccc(Cl)cc1C(=O)O)Nc1cccc(-c2ccoc2)c1]

Angstrom6 (A6 Peptide) is an 8 amino-acid peptide derived from single-chain urokinase plasminogen activator (scuPA) and interferes with the uPA/uPAR cascade and abrogates downstream effects. Angstrom6 binds to CD44 resulting in the inhibition of migration, invasion, and metastasis of tumor cells, and the modulation of CD44-mediated cell signaling.

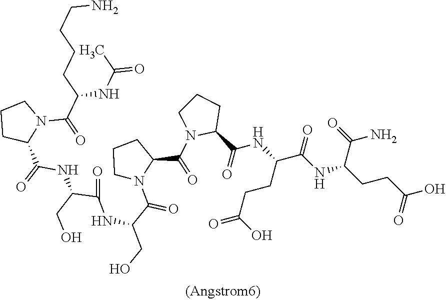 [SMILES: CC(=O)N[C@@H](CCCCN)C(=O)N1CCC[C@H]1C(=O)N[C@@H](CO)C(=O)N[C@@H](CO)C(=O)N1CCC[C@H]1C(=O)N1CCC[C@H]1C(=O)N[C@@H](CCC(=O)O)C(=O)N[C@@H](CCC(=O)O)C(N)=O]

Upamostat (WX-671) is a serine protease inhibitor. Upamostat is the orally available prodrug of the WX-UK1, which is a uPA inhibitor.

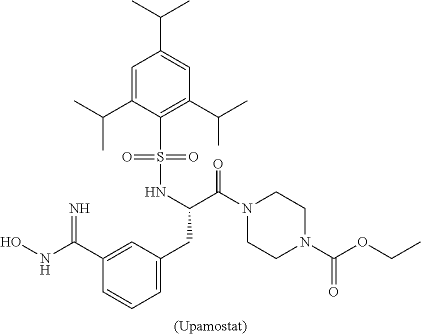 [SMILES: CCOC(=O)N1CCN(C(=O)[C@H](Cc2cccc(C(=N)NO)c2)NS(=O)(=O)c2c(C(C)C)cc(C(C)C)cc2C(C)C)CC1]

TM5275 sodium is a PAI-1 with an IC50 of 6.95 μM.

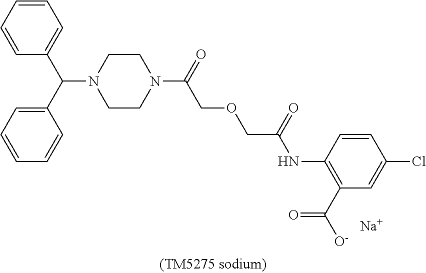 [SMILES: O=C(COCC(=O)N1CCN(C(c2ccccc2)c2ccccc2)CC1)Nc1ccc(Cl)cc1C(=O)[O-] ; [Na+]]

Loureirin B (Standard) is the analytical standard of Loureirin B. This product is intended for research and analytical applications. Loureirin B, a flavonoid extracted from Dracaena cochinchinensis, is an inhibitor of PAI-1, with an IC50 of 26.10 μM; Loureirin B also inhibits KATP, the phosphorylation of ERK and JNK, and has anti-diabetic activity.

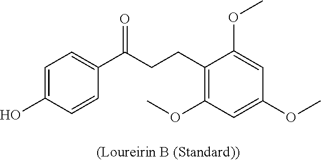 [SMILES: COc1cc(OC)c(CCC(=O)c2ccc(O)cc2)c(OC)c1]

PPACK dihydrochloride is a plasminogen activator inhibitor. PPACK dihydrochloride can inhibit changes in fibrin degradation products, plasminogen and alpha 2-antiplasmin. PPACK dihydrochloride also inhibits the binding of rt-PA to plasma protease inhibitors.

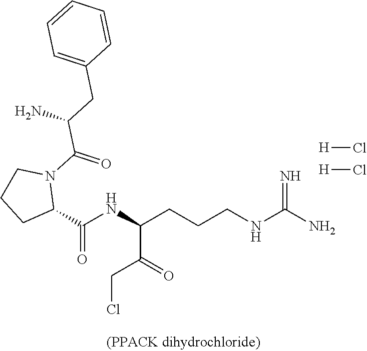

MDI-2268 is an inhibitor of PAI-1. MDI-2268 has good antithrombotic properties and regulates blood coagulation and fibrinolysis process by enhancing fibrinolysis. MDI-2268 can be used in research areas such as deep vein thrombosis.

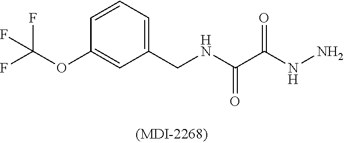 [SMILES: NNC(=O)C(=O)NCc1cccc(OC(F)(F)F)c1]

Aleplasinin is an orally active, potent, BBB-penetrated and selective PAI-1 inhibitor. Aleplasinin increases amyloid-β (Aβ) catabolismand ameliorates amyloid-related pathology. Aleplasinin improves memory deficiency. Aleplasinin can be used for Alzheimer's disease research.

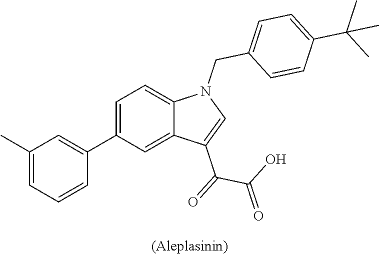 [SMILES: Cc1cccc(-c2ccc3c(c2)c(C(=O)C(=O)O)cn3Cc2ccc(C(C)(C)C)cc2)c1]

UKI-1 (WX-UK1) is a potent uPA inhibitor with a Ki of 0.41 μM. UKI-1 is also a low molecular weight serine protease inhibitor. UKI-1 is a potent antimetastatic agent and inhibits the invasive capacity of carcinoma cells.

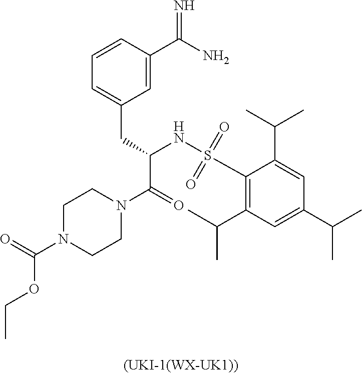 [SMILES: CCOC(=O)N1CCN(C(=O)[C@H](Cc2cccc(C(=N)N)c2)NS(=O)(=O)c2c(C(C)C)cc(C(C)C)cc2C(C)C)CC1]

UK-371804 is a uPA inhibitor with a Ki of 10 nM.

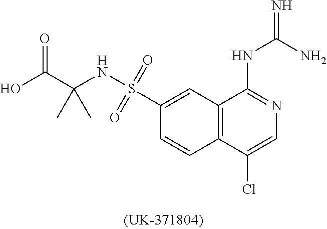 [SMILES: CC(C)(NS(=O)(=O)c1ccc2c(Cl)cnc(NC(=N)N)c2c1)C(=O)O]

PPACK TFA is a plasminogen activator and thrombin inhibitor. PPACK TFA can inhibit changes in fibrin degradation products, plasminogen and alpha 2-antiplasmin. PPACK TFA also inhibits the binding of rt-PA to plasma protease inhibitors.

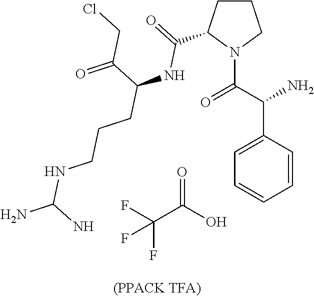

CDE-096 is a potent inhibitor of PAI-1. CDE-096 prevents PAI-1 from inactivating tPA and uPA with similar potency (IC50-30 and 25 nM, respectively) and is active against glycosylated PAI-1, as well as PAI-1 derived from several species (IC50=19, 22 and 18 nM for murine, rat, and Porcine PAI-1, respectively).

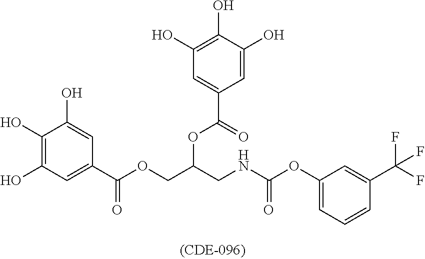 [SMILES: O=C(NCC(COC(=O)c1cc(O)c(O)c(O)c1)OC(=O)c1cc(O)c(O)c(O)c1)Oc1cccc(C(F)(F)F)c1]

Anecortave acetate is a potent ocular angiostatic agent. Anecortave acetate inhibits neovascularization which is induced by many different angiogenic factors, and increases plasminogen activator inhibitor-1 (PAI-1) mRNA expression. Anecortave acetate can be used to research ocular neovascular diseases.

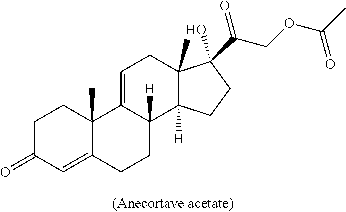

Diaplasinin (PAI-749) is a plasminogen activator inhibitor-1 (PAI-1) inhibitor with IC50 of 295 nM. Diaplasinin has antithrombotic efficacy.

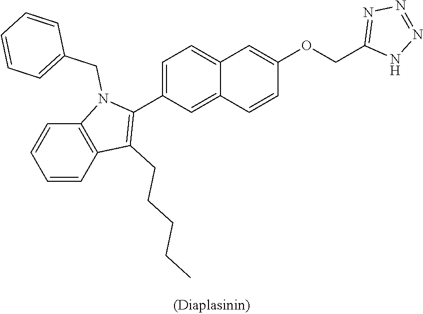 [SMILES: CCCCCc1c(-c2ccc3cc(OCc4nnn[nH]4)ccc3c2)n(Cc2ccccc2)c2ccccc12]

Loureirin B, a flavonoid extracted from Dracaena cochinchinensis, is an inhibitor of plasminogen activator inhibitor-1 (PAI-1), with an IC50 of 26.10?μM; Loureirin B also inhibits KATP, the phosphorylation of ERK and JNK, and has anti-diabetic activity.

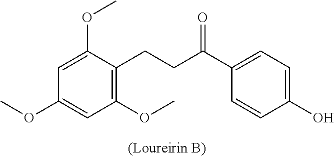 [SMILES: COc1cc(OC)c(CCC(=O)c2ccc(O)cc2)c(OC)c1]

ZK824859 hydrochloride is an oral available and selective uPA inhibitor with IC50s of 79 nM, 1580 nM and 1330 nM for human uPA, tPA, and plasmin, respectively.

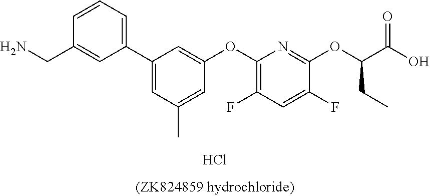 [SMILES: CC[C@@H](Oc1nc(Oc2cc(C)cc(-c3cccc(CN)c3)c2)c(F)cc1F)C(=O)O ; Cl]

Toddalolactone, a main component of Toddalia asiatica, inhibits the activity of recombinant human PAI-1, with an IC50 value of 37.31 μM.

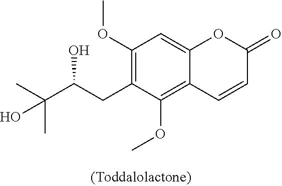 [SMILES: COc1cc2oc(=O)ccc2c(OC)c1C[C@@H](O)C(C)(C)O]

BC-11 hydrobromide is a selective TMPRSS2 inhibitor (TMPRSS2 is a key host cellular factor for viral entry and SARS-COV-2 pathogenesis), and a selective uPA inhibitor (IC50=8.2 μM). BC-11 hydrobromide is cytotoxic to triple-negative MDA-MB231 breast cancer cells. BC-11 hydrobromide is used in research on viral infections and cancer.

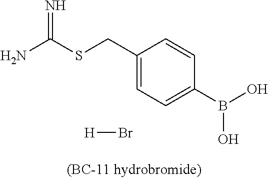

ZK824190 hydrochloride is an orally available and selective uPA inhibitor as a potential treatment for multiple sclerosis. IC50s of 237, 1600 and 1850 nM for uPA, tPA, and Plasmin, respectively.

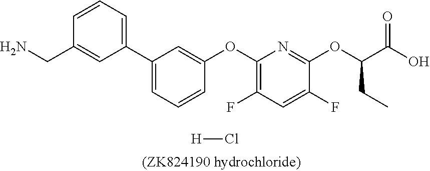

SK-216 is a PAI-1 inhibitor that acts as an anti-metastatic agent for human osteosarcoma and inhibits lung metastasis of human osteosarcoma.

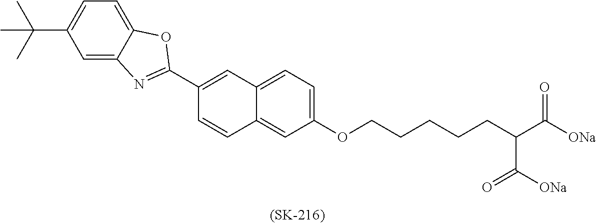

ZK824190 is an orally available and selective uPA inhibitor as a potential treatment for multiple sclerosis. IC50s of 237, 1600 and 1850 nM for uPA, tPA, and Plasmin, respectively.

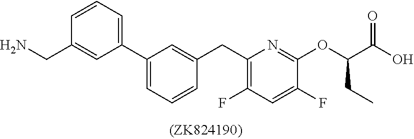 [SMILES: CC[C@@H](Oc1nc(Cc2cccc(-c3cccc(CN)c3)c2)c(F)cc1F)C(=O)O]

AZ3976 is a potent PAI-1 inhibitor with an IC50 value of 26 μM in an enzymatic chromogenic assay. AZ3976 is active with an IC50 of 16 μM in a plasma clot lysis assay. AZ3976 does not bind to active PAI-1 but bound reversibly to latent PAI-1. AZ3976 inhibits PAI-1 by enhancing the latency transition of active PAI-1. AZ3976 displays profibrinolytic activities in a human plasma clot lysis assay.

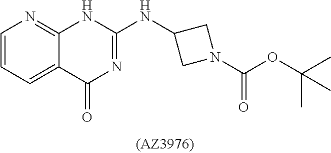 [SMILES: CC(C)(C)OC(=O)N1CC(Nc2nc(=O)c3cccnc3[nH]2)C1]

UCD38B hydrochloride is a cell permeant, competitive enzymatic uPA inhibitor with an IC50 value of 7 μM. UCD38B hydrochloride targets intracellular uPA causing mistrafficking of uPA into perinuclear mitochondria, reducing the mitochondrial membrane potential, and followed by the release of apoptotic inducible factor (AIF). UCD38B hydrochloride induces apoptosis.

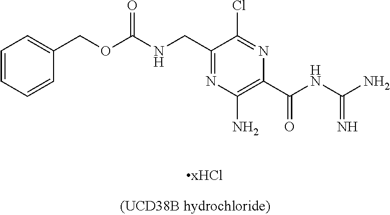 [SMILES: N=C(N)NC(=O)c1nc(Cl)c(CNC(=O)OCc2ccccc2)nc1N]

TM5441 is an orally bioavailable inhibitor of PAI-1, has IC50 values between 13.9 and 51.1 μM. TM5441 induces intrinsic apoptosis in several human cancer cell lines. TM5441 attenuates Nω-nitro-1-arginine methyl ester-induced cardiac hypertension and vascular senescence.

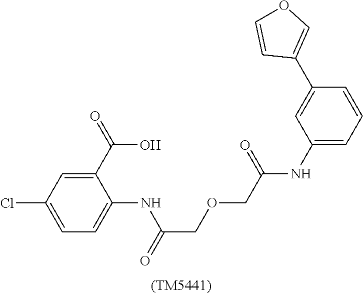

Geodin, a fungal metabolite, shows antibacterial activity. Geodin also is an inhibitor of PAI-1.

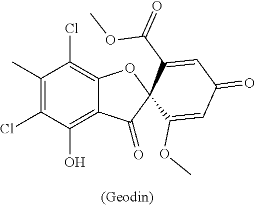 [SMILES: COC(=O)C1=CC(=O)C=C(OC)[C@]12Oc1c(Cl)c(C)c(Cl)c(O)c1C2=O]

GGACK (H-Glu-Gly-Arg-CMK) is an irreversible substrate-like serine protease uPA inhibitor.

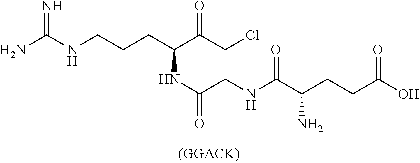 [SMILES: N=C(N)NCCC[C@H](NC(=O)CNC(=O)[C@@H](N)CCC(=O)O)C(=O)CCl]

ZK824859 is an oral available and selective uPA inhibitor with IC50s of 79 nM, 1580 nM and 1330 nM for human uPA, tPA, and plasmin, respectively.

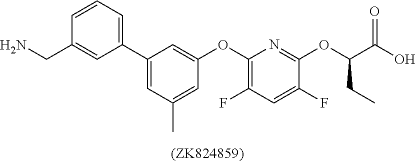 [SMILES: CC[C@@H](Oc1nc(Oc2cc(C)cc(-c3cccc(CN)c3)c2)c(F)cc1F)C(=O)O]

28-O-β-D-Glucopyranosyl pomolic acid is a uPA inhibitor with IC50 at 37.82 μM.

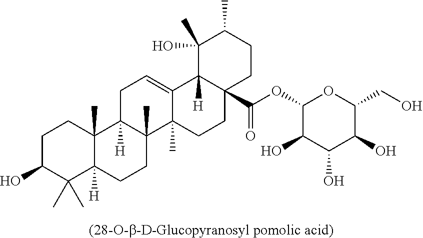

PPACK is a plasminogen activator inhibitor. PPACK can inhibit changes in fibrin degradation products, plasminogen and alpha 2-antiplasmin. PPACK also inhibits the binding of rt-PA to plasma protease inhibitors.

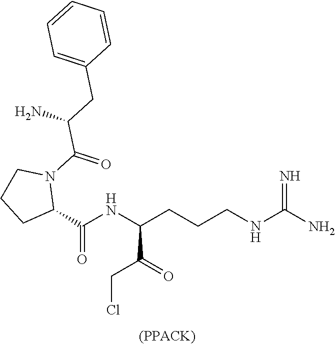 [SMILES: N=C(N)NCCC[C@H](NC(=O)[C@@H]1CCCN1C(=O)[C@H](N)Cc1ccccc1)C(=O)CCl]

Fendosal (HP 129) is an orally active, potent non-steroidal anti-inflammatory agent. Fendosal (HP 129) is also an inhibitor of PAI-1.

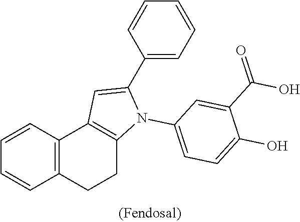 [SMILES: O=C(O)c1cc(-n2c(-c3ccccc3)cc3c2CCc2ccccc2-3)ccc1O]

Glutaryl-Gly-Arg-AMC is a peptide substrate of uPA.

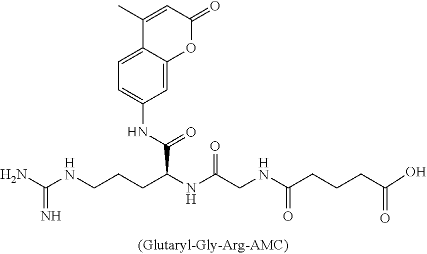 [SMILES: Cc1cc(=O)oc2cc(NC(=O)[C@H](CCCNC(=N)N)NC(=O)CNC(=O)CCCC(=O)O)ccc12]

UK-371804 hydrochloride (compound 24) is a uPA inhibitor with a Ki of 10 nM.

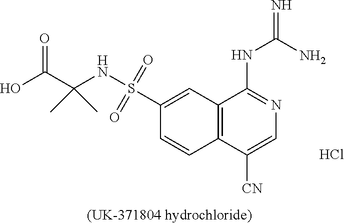 [SMILES: CC(C)(NS(=O)(=O)c1ccc2c(C#N)cnc(NC(=N)N)c2c1)C(=O)O ; Cl]

CJ-463 is a potent and selective uPA inhibitor. CJ-463 has antitumor activity.

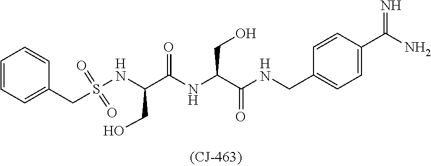 [SMILES: N=C(N)c1ccc(CNC(=O)[C@H](CO)NC(=O)[C@@H](CO)NS(=O)(=O)Cc2ccccc2)cc1]

TM5007 is a potent and orally active inhibitor of PAI-1 with an IC50 of 29 μM. TM5007 enhance fibrinolysis activity and inhibits coagulation. TM5007 also prevents the fibrotic process initiated by bleomycin in mouse lung.

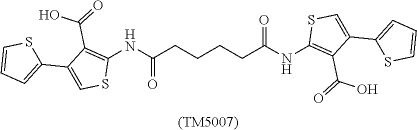

### Route of Administration

The PAI-1 activity modifier of the present application may be administered to the subject with known methods, such as intravenous administration as a bolus or by continuous infusion over a period of time, intramuscular administration, subcutaneous administration, and intra-peritoneal or oral administration. In certain embodiments, the PAI-1 activity modifiers is administered directly to gastrointestinal tissue, including administration directly to the tissue surface during invasive procedures such as endoscopy.

PAI-1 activity modifiers can be administered in the usual pharmaceutically acceptable carriers. Acceptable carriers include, but are not limited to, saline, buffered saline, glucose in saline. Solid supports, liposomes, nanoparticles, microparticles, nanospheres or microspheres may also be used as carriers for administration of the PAI-1 activity modifiers.

One or more of the PAI-1 activity modifiers discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

The therapeutic compositions administered by these methods are administered directly or indirectly into the environment of the gastrointestinal tissue undergoing caustic damage. Conventional and pharmaceutically acceptable routes of administration include, but are not limited to, systemic routes, such as intravenous, intramuscular, or subcutaneous, and other parenteral routes of administration. Routes of administration may be combined, if desired. In some embodiments, the administration is repeated periodically.

These therapeutic compositions may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

### Dosage

The appropriate dosage (“therapeutically effective amount”) of a PAI-1 activity modifier of the present application will depend, for example, on the condition to be treated, the severity and course of the condition, whether the PAI-1 activity modifier is administered for preventive or therapeutic purposes, previous therapy, the patient's clinical history and response to the PAI-1 activity modifier, the type of PAI-1 activity modifier used, and the discretion of the attending physician. The PAI-1 activity modifier is suitably administered to the patent at one time or over a series of treatments and may be administered to the patent at any time from diagnosis onwards. The PAI-1 activity modifier may be administered as the sole treatment or in conjunction with other drugs or therapies useful in treating the condition in question.

As a general proposition, the therapeutically effective amount of the PAI-1 activity modifier, will be administered in the range of about 0.05 mg/kg body weight/day to about 250 mg/kg body weight/day whether by one or more administrations. In particular embodiments, the range of the PAI-1 activity modifier administered is from about 0.5 mg/kg body weight/day to about 100 mg/kg body weight/day, from about 1 mg/kg body weight/day to about 50 mg/kg body weight/day, or from about 5 mg/kg body weight/day to about 25 mg/kg body weight/day.

In some embodiments, the daily dose of the PAI-1 activity modifier of the present application is in the range of 0.01-5000 mg, 0.1-5000 mg, 1-5000 mg, 10-5000 mg, 100-5000 mg, 1000-5000 mg, 0.01-1000 mg, 0.1-1000 mg, 1-1000 mg, 10-1000 mg, 100-1000 mg, 0.01-100 mg, 0.1-100 mg, 1-100 mg, 10-100 mg, 0.01-10 mg, 0.1-10 mg, 1-10 mg, 0.01-1 mg, 0.1-1 mg, or 0.01-0.1 mg.

The PAI-1 activity modifier of the present application may be administered, as appropriate or indicated, a single dose as a bolus or by continuous infusion, or as multiple doses by bolus or by continuous infusion. Multiple doses may be administered, for example, multiple times per day, once daily, every 2, 3, 4, 5, 6 or 7 days, weekly, every 2, 3, 4, 5 or 6 weeks or monthly. However, other dosage regimens may be useful. The progress of this therapy is easily monitored by conventional techniques.

The dosages and treatment regimens utilizing PAI-1 activity modifier of the present application can be determined by the person of skill in the art. Certain of the PAI-1 activity modifiers of the present application are approved for use for the treatment of other conditions, and thus dosages and prescribing information is known.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the present application, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Formulations

In some embodiments, the PAI-1 activity modifier of the present application is formulated for the desired route of administration using one or more pharmaceutically acceptable carriers. As used herein the language “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the application is formulated to be compatible with its intended route of administration. Examples of routes of administration include parenteral, e.g., intrathecal, intra-arterial, intravenous, intradermal, subcutaneous, oral, transdermal (topical) and transmucosal administration. In certain embodiments, the pharmaceutical composition is administered directly into a damaged or injured tissue.

Solutions or suspensions used for parenteral, intradermal, or subcutaneous application can include the following components: a sterile diluent such as water for injection, saline solution, fixed oils, polyethylene glycols, glycerin; propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active, ingredient plus any additional desired ingredient from a previously sterile-filtered solution thereof.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially, for example, from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the application are dictated by and directly dependent on the unique characteristics of the active compound and the particular therapeutic effect to be achieved, and the limitations inherent in the art of compounding such an active compound for the treatment of individuals.

### Combination Therapy

In some embodiments, the method of the present application further comprises administering to the subject one or more pharmaceutical compositions comprising a steroid, anti-inflammatory, and/or an antibiotic agent. In some embodiments, a pharmaceutical composition is administered orally, intramuscularly, subcutaneously or intravenously.

In some embodiments, an anti-inflammatory agent is selected from aminosalicylates (such as mesalamine or olsalazine), corticosteroids (such as prednisolone or budesonide), immunosuppressants (such as azathioprine or mycophenolate mofetil), or advanced biologics (such as anti-TNF, anti-IL-17, or anti-IL-17R).

In some embodiments, the method of the present application further comprises administering to the subject one or more anti-fibrotic agents. Exemplary anti-fibrotic agents include, but are not limited to, pirfenidone, nintedanib, and nerandomilast.

An aspect of the present application is related to a method of preventing and/or treating gastrointestinal damage in a subject, comprising the step of administering to the subject an effective amount of a pharmaceutical composition comprising a PAI-1 activity modifier.

In some embodiments, the PAI-1 activity modifier is a PAI-1 inhibitor. In some embodiments, the PAI-1 activity modifier is selected from the group consisting of: tiplaxtinin, aleplasinin (PAZ-417), anecortave acetate, angstrom6, AZ3976, BC-11 hydrobromide, CDE-096, CJ-463, diaplasinin (PAI-749), fendosal (HP 129), glutaryl-Gly-Arg-AMC, GGACK (H-Glu-Gly-Arg-CMK), geodin, loureirin B (Standard), PPACK, PPACK dihydrochloride, PPACK TFA, MDI-2268, SK-216, TM5007, TM5275, TM5275 sodium, TM5441, upamostat, UKI-1, UCD38B hydrochloride, toddalolactone, UK-371804, UK-371804 hydrochloride (compound 24), ZK824859, ZK824859 hydrochloride, ZK824190, ZK824190 hydrochloride, 28-O-β-D-glucopyranosyl pomolic, TM5614, S35225, MDI-2517, IMD-1622, and WAY-140312.

In some embodiments, the PAI-1 activity modifier is a plasminogen activator.

In some embodiments, the PAI-1 activity modifier is selected from the group consisting of: tissue plasminogen activator, recombinant tissue plasminogen activator, urokinase plasminogen activator, recombinant urokinase plasminogen activator, bat salivary plasminogen activator, streptokinase, staphylokinase, kallikreins, and Factor XIIa. In some embodiments, the tissue plasminogen activator is alteplase, tenecteplase, or reteplase.

In some embodiments, the subject has gastrointestinal damage.

In some embodiments, the gastrointestinal damage is an esophageal, stomach, intestinal, or tongue stricture.

In some embodiments, the gastrointestinal damage is caustic damage.

In some embodiments, the gastrointestinal damage is from thermal injury, inflammation, scarring, chronic acid reflux (GERD), radiation therapy, NSAIDs, antibiotics, eosinophilic esophagitis, tumors, or congenital issues.

In some embodiments, the PAI-1 activity modifier is administered orally, intravenously, intramuscularly, or subcutaneously. In some embodiments, the PAI-1 activity modifier is administered at a daily dose of 0.5-250 mg/kg body weight. In some embodiments, the PAI-1 activity modifier is administered at a daily dose of 5-25 mg/kg body weight.

In some embodiments, the method further comprises the step of: administering to the subject a steroid, antibiotic, anti-inflammatory agent, immunosuppressant, anti-fibrotic agent, or combinations thereof.

An aspect of the present application relates to a method for reducing gastrointestinal inflammation in a subject, comprising the step of administering to the subject an effective amount of a pharmaceutical composition comprising a PAI-1 activity modifier.

In some embodiments, the gastrointestinal inflammation is due to caustic damage, thermal injury, inflammation, scarring, chronic acid reflux (GERD), radiation therapy, NSAIDS, antibiotics, eosinophilic esophagitis, tumors, or congenital issues.

An aspect of the present application relates to a method for reducing gastrointestinal scar formation in a subject, comprising the step of administering to the subject an effective amount of a pharmaceutical composition comprising a PAI-1 activity modifier.

In some embodiments, the gastrointestinal scar formation is due to caustic damage, thermal injury, inflammation, scarring, chronic acid reflux (GERD), radiation therapy, NSAIDs, antibiotics, eosinophilic esophagitis, tumors, or congenital issues.

An aspect of the present application relates to a method for rejuvenating or reconstituting damaged tissue in a subject suffering from caustic injury, comprising the step of administering to the subject an effective amount of a pharmaceutical composition comprising a PAI-1 activity modifier.

An aspect of the present application relates to a method for preventing or treating a stricture in a subject, comprising administering to the subject an effective amount of a pharmaceutical composition comprising a PAI-1 activity modifier.

In some embodiments, the stricture is an esophageal, stomach, or tongue stricture.

In some embodiments, the stricture is a caustic stricture.

An aspect of the present application relates to a method for down-regulating one or more genes that is up-regulated by exposure of a subject to a caustic substance comprising, administering to the subject an effective amount of a pharmaceutical composition comprising a PAI-1 activity modifier. In some embodiments, the one or more genes include at least one of SERPINE1, Serpina3n, Tlr4, CD-24a, Myl2, Sod1, Atp6v0d2, Ly6g2, Neurod1, Fabp3, Nqo1, Peds1, Folr1, Lbh, Caps1, Gsto1, Glu1, Col4a3, and IL-17RE.

An aspect of the present application relates to a method for up-regulating one or more genes that is down-regulated by exposure of a subject to a caustic substance comprising, administering to the subject an effective amount of a pharmaceutical composition comprising a PAI-1 activity modifier. In some embodiments, the one or more genes include at least one of Actc1, Atp2a1, Tlr6, Tlr12, CD-28, Cd-47, CD-79a, CD-79b, Cnn1, Des, Foxp3, IL-21, IL-21R, IL-4, Nod2, Scn5a, TGF-β1, and Traf2.

The following examples are offered by way of illustration of certain embodiments of aspects of the application herein. None of the examples should be considered limiting on the scope of the application.

### EXAMPLES

### Example 1. Inhibition of PAI-1 in Mouse Model of CES

A mouse model has been developed of alkali injury to test the role of specific pathways that drive CES formation, and to evaluate potential therapeutic targets for this condition. Using the mouse model herein studies tested the hypothesis that PAI-1 is a driver of CES formation using our mouse model of alkali injury.

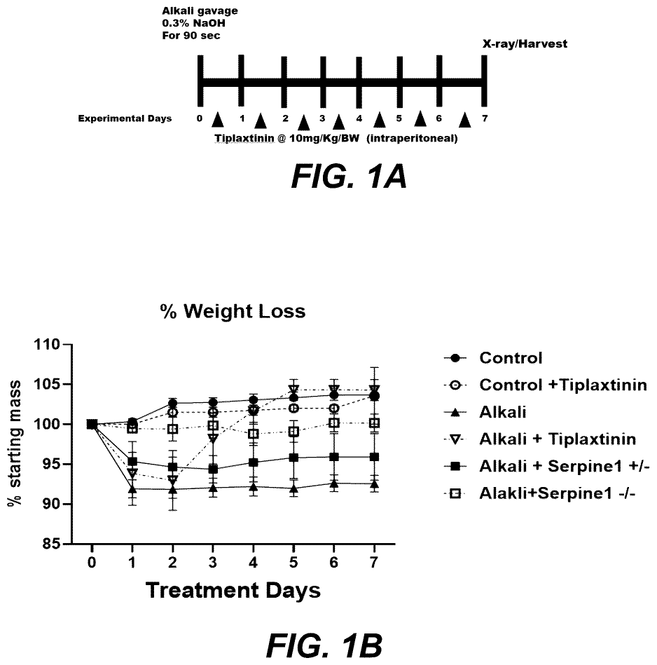

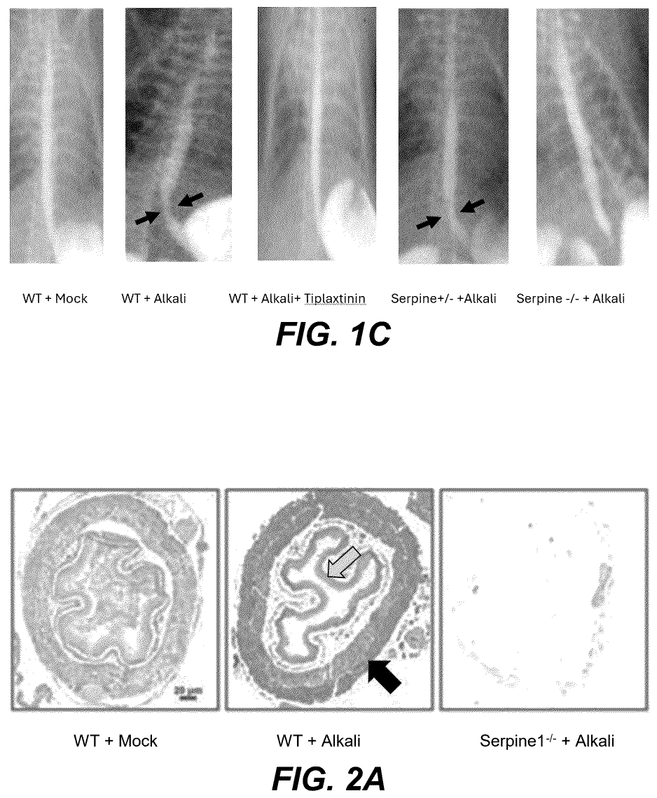

Anesthetized male and female C57/BL6J (wild-type (WT)), SERPINE1+/− and SERPINE1−/− mice aged 8 to 20 weeks mice were gavaged with 0.3% NaOH for 90 seconds via gastric balloon catheter on day 0. Sham controls were perfused with saline solution. Starting 6 hours post-gavage mice were given i.p. injections of tiplaxtininat 10 mg/kg body weight daily for 7 days (FIG. 1A). Experiments were repeated 2-4 times with 3-5 mice per group. The study evaluated cellular and molecular targets of alkali damage using histology and immunohistochemistry. Tiplaxtinin, a small molecule PAI-1 inhibitor, is shown here to have efficacy in the prevention of CES post alkali exposure in models of mouse colonic injury.

The study showed that PAI-1 inhibition can be an effective preventative treatment for strictures throughout the gastrointestinal tract in response to injury and inflammation.

### Example 2: Identification of Co-Expressed Genes

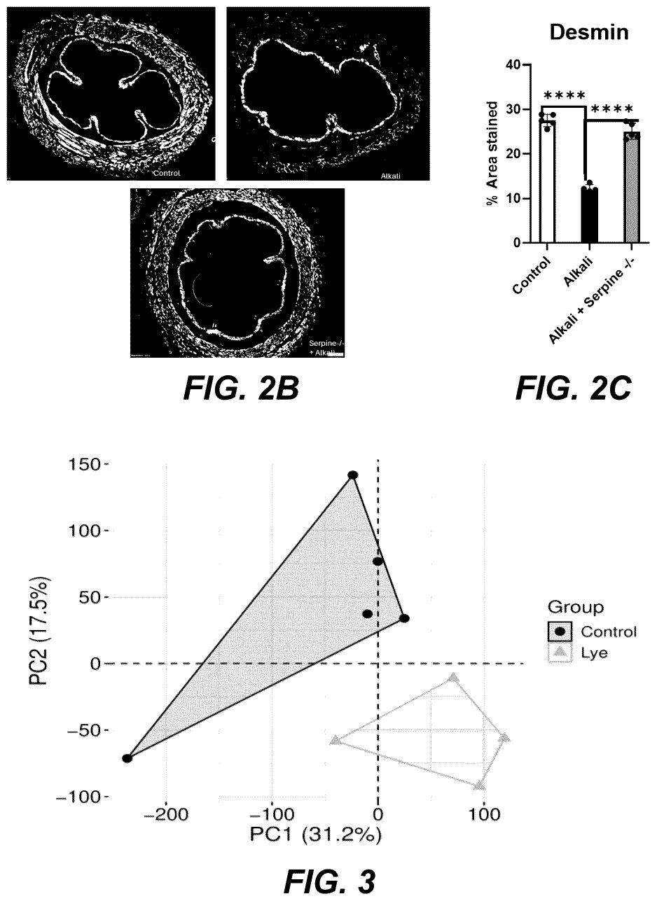

Post alkali exposure, WT mice lost weight compared to sham controls (15% of baseline body weight for days 2-7 post lye perfusion, FIG. 1B). Mice were subjected to Barium Swallow test on day 7 post alkali exposure and barium-contrast x-rays were performed. Wild Type (WT) mice with alkali treatment showed 64% ( 16/25) stricture and Serpine 1+/− mice showed 16% ( 2/12), while treatment with tiplaxtinin and Serpine1−/− did not show any stricture (FIG. 1C). Representative inverted grayscale photomicrographs of anti-PAI-1 immunofluorescence staining of esophageal sections from mice treated as indicated (n≥2 mice per group). Note stronger signal in both muscle (dark arrow) and epithelium (open arrow) 7 days following alkali instillation (FIG. 2A). Antibody specificity was confirmed by lack of signal in Serpine1−/− esophageal tissue. Esophageal tissue of alkali-exposed WT mice showed significant increase in PAI-1 expression, particularly in the muscularis propria (FIG. 2A) and the enteric nervous system (ENS). Sparse or absent ENS in the H&E-stained sections of the distal esophageal stricture, as well as decreased anti-TUJ immunoreactivity, correlated with loss of Desmin in the corresponding areas, consistent with denervation of the smooth muscle layer in this region (FIG. 2B). Quantification data shows decreased levels of Desmin in alkali treated mice while in Serpine−/− mice the decrement was less (FIG. 2C).

This study showed that Serpine1−/− and Serpine1+/− mice were protected from the effects of alkali injury. Serpine1−/− mice had no weight loss while Serpine1+/− mice had 5% weight loss at day 2 and recovered by day 7 (FIG. 1B). CES developed in none of the Serpine1−/− mice (n=8), and in 2 of 12 (16.6%) Serpine1+/− mice. In the Serpine1−/− mice, ENS and muscularis propria desmin expression were maintained. Therapeutic effects of PAI-1 inhibition via tiplaxtinin given 6 hours after alkali exposure showed prevention of CES formation in all animals and weight loss comparable to Serpine1−/− mice.

As seen here, pharmacological and genetic inhibition of PAI-1 ameliorated esophageal injuries and CES formation induced by alkali exposure. Together, the data indicate PAI-1 is a novel and attractive target for prevention of CES after alkali injury.

### Example 3: RNA Sequence Analysis

Eight-week old male C57BL/6J mice were perfused with 0.3% NaOH for 60 seconds to initiate CES formation. There were 2 groups, group 1 control and group 2 exposed to alkali. The animals were sacrificed 24 h post alkali exposure. Esophagi were removed and cut into proximal and other distal sections. Esophagus sections were individually suspended in 1 ml RNAlater™ (Invitrogen) and stored at −80° C. for further isolation. RNA was later extracted using the RNeasy Micro Kit (Qiagen) according to manufacturer's directions. Briefly, cells were centrifuged and lysed in RLT buffer with 2-mercaptoethanol. RNA was extracted and eluted in 14 μl of nuclease free water and stored at −80° C. RNA was extracted was assessed by Qubit (Thermo Fisher) and Tapestation (Agilent). Library prep was done with SMARTer® Stranded Total RNA-Seq Kit v3—Pico Input Mammalian (Takara Bio) and 50 million reads were obtained per sample. Sequencing datasets were produced using an Illumina NovaSeq 6000 instrument. To identify differentially expressed genes, DESeq2's median of ratios was used to normalize for sequencing depth and for calculation of fold changes and p-values (nominal & adjusted).

FIG. 3 shows principal component analysis (PCA) of the control and lye-treated groups. The PCA score plot shows more variability within the control group and less variability in the lye-treated group. The shift in the sample from the control to lye-treated, group indicating a measurable effect.

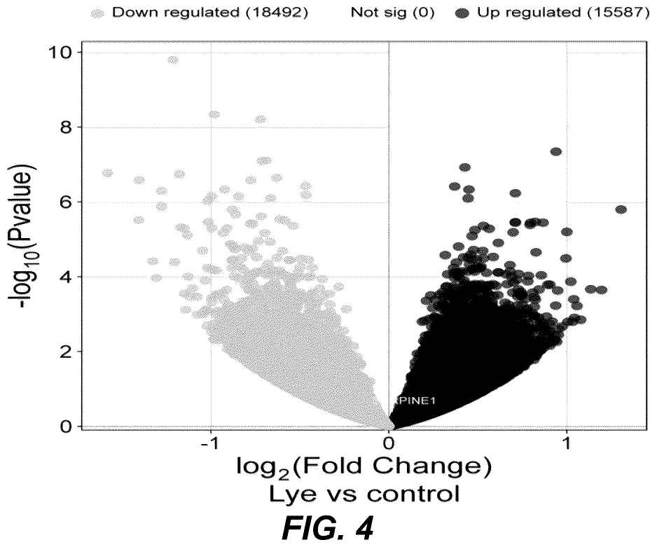

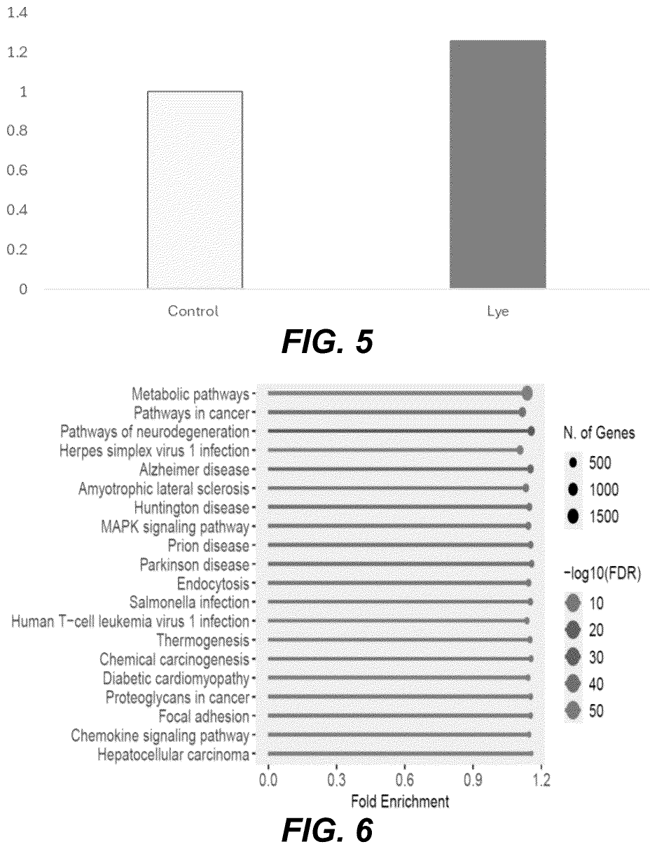

FIG. 4 shows a volcano plot of genes that are significantly altered between the control and lye-treated groups. The Volcano plot shows around 35,000 genes significantly altered between control and lye-treated group: 18,492 genes were downregulated and 15,587 genes were upregulated. All SERPIN family genes were upregulated in the lye-treated group, with SERPINE 1 upregulated by 1.25 fold at 24 hour after exposure (FIG. 5).

FIG. 6 shows a KEGG pathway graph of enrichment data related to PAI-1 expression in the control and lye-treated groups. Each row is a biological pathway. The top line represents the strongest pathway that is enriched in the gene list of the study. Consistent with the present findings of loss of enteric neuronal damage, the top 5 strongest in significance are the pathways related to neurodegenerative genes.

Table 1 is a list of genes whose expression is upregulated in the lye-treated group versus expression in controls.

TABLE 1

| | | Fold |
|---|---|---|
| Ensembl_ID | Name | Change |
| ENSMUSG00000037411 | SERPINE1 | 1.257103 |
| ENSMUSG00000021091 | Serpina3n | 89.81493 |
| ENSMUSG00000039005 | Tlr4 | 1.263124 |
| ENSMUSG00000047139 | Cd24a | 2.263521 |
| ENSMUSG00000013936 | Myl2 | 1.956886 |
| ENSMUSG00000022982 | Sod1 | 1.408199 |
| ENSMUSG00000028238 | Atp6v0d2 | 8.277186 |
| ENSMUSG00000047728 | Ly6g2 | 3.580737 |
| ENSMUSG00000034701 | Neurod1 | 3.477126 |
| ENSMUSG00000028773 | Fabp3 | 3.322761 |
| ENSMUSG00000003849 | Ngo1 | 3.118259 |
| ENSMUSG00000090213 | Peds1 | 3.139912 |
| ENSMUSG00000001827 | Folr1 | 3.107685 |
| ENSMUSG00000024063 | Lbh | 2.834234 |
| ENSMUSG00000039676 | Capsl | 2.788881 |
| ENSMUSG00000025068 | Gsto1 | 2.648268 |
| ENSMUSG00000026473 | Glul | 2.481746 |
| ENSMUSG00000079465 | Col4a3 | 1.487917 |
| ENSMUSG00000043088 | IL-17RE | 1.504681 |

Table 2 is a list of genes whose expression is downregulated in the lye-treated group versus expression in controls.

TABLE 2

| | | Fold |
|---|---|---|
| Ensembl_ID | Name | Change |
| ENSMUSG00000068614 | Actc1 | 0.723183 |
| ENSMUSG00000030730 | Atp2a1 | 0.707275 |
| ENSMUSG00000051498 | Tlr6 | 0.724366 |
| ENSMUSG00000062545 | Tlr12 | 0.722914 |
| ENSMUSG00000026012 | CD-28 | 0.267469 |
| ENSMUSG00000055447 | CD-47 | 0.925613 |
| ENSMUSG00000003379 | CD-79a | 0.392565 |
| ENSMUSG00000040592 | CD-79b | 0.396628 |
| ENSMUSG00000001349 | Cnn1 | 0.723834 |
| ENSMUSG00000026208 | Des | 0.685038 |
| ENSMUSG00000039521 | Foxp3 | 0.348316 |
| ENSMUSG00000027718 | IL-21 | 0.494965 |
| ENSMUSG00000030745 | IL-21R | 0.294187 |
| ENSMUSG00000000869 | IL-4 | 0.769189 |
| ENSMUSG00000055994 | Nod2 | 0.827726 |
| ENSMUSG00000032511 | Scn5a | 0.552784 |
| ENSMUSG00000002603 | TGF-β1 | 0.81362 |
| ENSMUSG00000026942 | Traf2 | 0.992664 |

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the object of the present application, and it is not intended to detail all those obvious modifications and variations of it which will become apparent to the skilled worker upon reading the description. It is intended, however, that all such obvious modifications and variations be included within the scope of the present application, which is defined by the following claims. The aspects and embodiments are intended to cover the components and steps in any sequence, which is effective to meet the objectives there intended, unless the context specifically indicates the contrary.

What is claimed is:

1. A method of preventing and/or treating gastrointestinal damage in a subject, comprising the step of administering to the subject an effective amount of a pharmaceutical composition comprising a PAI-1 activity modifier.

2. The method of claim 1, wherein the PAI-1 activity modifier is a PAI-1 inhibitor.

3. The method of claim 1, where the PAI-1 activity modifier is selected from the group consisting of: tiplaxtinin, aleplasinin (PAZ-417), anecortave acetate, angstrom6, AZ3976, BC-11 hydrobromide, CDE-096, CJ-463, diaplasinin (PAI-749), fendosal (HP 129), glutaryl-Gly-Arg-AMC, GGACK (H-Glu-Gly-Arg-CMK), geodin, loureirin B (Standard), PPACK, PPACK dihydrochloride, PPACK TFA, MDI-2268, SK-216, TM5007, TM5275, TM5275 sodium, TM5441, upamostat, UKI-1, UCD38B hydrochloride, toddalolactone, UK-371804, UK-371804 hydrochloride (compound 24), ZK824859, ZK824859 hydrochloride, ZK824190, ZK824190 hydrochloride, 28-O-β-D-glucopyranosyl pomolic, TM5614, S35225, MIDI-2517, IMD-1622, and WAY-140312.

4. The method of claim 1, wherein the PAI-1 activity modifier is a plasminogen activator.

5. The method of claim 1, wherein the PAI-1 activity modifier is selected from the group consisting of: tissue plasminogen activator, recombinant tissue plasminogen activator, urokinase plasminogen activator, recombinant urokinase plasminogen activator, bat salivary plasminogen activator, streptokinase, staphylokinase, kallikreins, and Factor XIIa.

6. The method of claim 5, wherein the tissue plasminogen activator is alteplase, tenecteplase, or reteplase.

7. The method of claim 1, wherein the subject has gastrointestinal damage.

8. The method of claim 7, wherein the gastrointestinal damage is an esophageal, stomach, intestinal, or tongue stricture.

9. The method of claim 7, wherein the gastrointestinal damage is caustic damage.

10. The method of claim 7, wherein the gastrointestinal damage is from thermal injury, inflammation, scarring, chronic acid reflux (GERD), radiation therapy, NSAIDs, antibiotics, eosinophilic esophagitis, tumors, or congenital issues.

11. The method of claim 1, wherein the PAI-1 activity modifier is administered orally, intravenously, intramuscularly, or subcutaneously.

12. The method of claim 1, wherein the PAI-1 activity modifier is administered at a daily dose of 0.5-250 mg/kg body weight.

13. The method of claim 1, wherein the PAI-1 activity modifier is administered at a daily dose of 5-25 mg/kg body weight.

14. The method of claim 1, further comprising the step of: administering to the subject a steroid, antibiotic, anti-inflammatory agent, immunosuppressant, anti-fibrotic agent, or combinations thereof.

15. A method for reducing gastrointestinal inflammation in a subject, comprising the step of administering to the subject an effective amount of a pharmaceutical composition comprising a PAI-1 activity modifier.

16. The method of claim 15, wherein the gastrointestinal inflammation is due to caustic damage.

17. The method of claim 15, wherein the gastrointestinal inflammation is due to thermal injury, inflammation, scarring, chronic acid reflux (GERD), radiation therapy, NSAIDs, antibiotics, eosinophilic esophagitis, tumors, or congenital issues.

18. A method for reducing gastrointestinal scar formation in a subject, comprising the step of administering to the subject an effective amount of a pharmaceutical composition comprising a PAI-1 activity modifier.

19. The method of claim 18, wherein the gastrointestinal scar formation is due to caustic damage.

20. The method of claim 18, wherein the gastrointestinal scar formation is due to thermal injury, inflammation, scarring, chronic acid reflux (GERD), radiation therapy, NSAIDs, antibiotics, eosinophilic esophagitis, tumors, or congenital issues.
